(19) United States
(12) Patent Application Publication
(10) Pub. No.: US 2002/0165286 A1
(43) Pub. Date: Nov. 7, 2002

(54) Dermal anti-inflammatory composition

(76) Inventors: Hanne Hedeman, Vaelose (DK); Pia Klie Refer, Helsingor (DK); Erik Johannes Didriksen, Ballerup (DK); Ann Vivian Fullerton, Vanlose (DK); Helle Aaes, Birkerod (DK); Lotte Groth, Roskilde (DK)

Correspondence Address: BIRCH STEWART KOLASCH & BIRCH, PO BOX 747, FALLS CHURCH, VA, 22040-0747, US

(21) Appl. No.: 10/003,709

(22) Filed: Dec. 6, 2001

Related U.S. Application Data
(60) Provisional application No. 60/251,882, filed on Dec. 8, 2000.

(51) Int. Cl. A61K 47/00

(52) U.S. Cl. 514/785

(57) ABSTRACT

A pharmaceutical composition for dermal application comprises a lipophilic anti-inflammatory compound and a pharmaceutically acceptable vehicle comprising a lipophilic excipient capable of solubilizing the anti-inflammatory compound and targeting said compound to the pilosebaceous ducts on application of the composition on the skin. The composition may be used in the treatment of dermal inflammatory conditions, in particular acne.

### FIELD OF INVENTION

The present invention relates to a pharmaceutical composition for dermal application of a lipophilic anti-inflammatory compound, and the use of said composition in a method of treating inflammatory conditions of the skin, in particular acne.

### BACKGROUND OF THE INVENTION

Acne is a skin condition which is a multifactorial disease affecting the pilosebaceous follicles, characterised by increased sebum production and release of sebum from the sebaceous glands, the presence of excessive amounts of sebum in the duct of the pilosebaceous follicles leading to the formation of comedones (solidified sebum plugs in the follicular duct). Further closing of the ducts results in the formation of pustules, papules or cysts which are often subject to bacterial colonisation, especially by Propionibacterium acnes, and localised inflammation. Acne vulgaris is the most common skin disorder among teenagers, but substantial numbers of adults aged 20-40 are also affected by acne. Currently available drugs for the treatment of acne include benzoyl peroxide, azelaic acid, topical and systemic antibiotics, such as Fucidin®, clindamycin, erythromycin, and tetracycline retinoids, such as adapalene, tretinoin, isotretinoin, and hormones, such as estrogen. There are, however, serious drawbacks with these medications including teratogenicity, skin irritation, photosensibilisation, etc., cf. Table 1 below.

Because of the negative psychosocial consequences for affected individuals, and the relatively limited numbers of drugs available for topical treatment of acne and the severity of the known side effects of these drugs, the provision of new medicaments for adequate therapy of acne is very important.

The present inventors have found that selected aminobenzophenones are effective anti-acne agents in both in vitro and in vivo models of acne and acne related disorders.

It is an object of the invention to provide topical compositions which have a satisfactory effect on acne and related skin conditions and which give rise to significantly less skin irritation and other adverse effects than the commercial preparations e.g. containing retinoids as active compounds.

### SUMMARY OF THE INVENTION

While aminobenzophenones have been found to have an excellent effect on acne and other inflammatory skin conditions on systemic (e.g. oral) administration, it is in many cases preferred when treating inflammatory skin conditions to provide the active compound in a topical preparation to avoid or reduce any adverse systemic effects of the active compounds. The object of the present invention is therefore to provide a formulation in which a lipophilic active compound may be dissolved or solubilised, and which permits targeting of the active compound on application to areas of the skin where a local anti-inflammatory effect would be beneficial.

Accordingly, the present invention relates to a pharmaceutical composition for dermal application comprising a lipophilic anti-inflammatory compound and a pharmaceutically acceptable vehicle comprising a lipophilic excipient capable of solubilising the anti-inflammatory compound and targeting said compound to the pilosebaceous ducts on application of the composition on the skin.

In the present context, the term “pharmaceutically acceptable” is intended to indicate that the vehicle and individual components thereof are of sufficient purity and are suitable for use in human skin and tissues without causing undue toxicity, irritation, allergic response or the like.

In another aspect, the invention relates to the use of a pharmaceutical composition comprising a lipophilic anti-inflammatory compound and a pharmaceutically acceptable vehicle comprising a lipophilic excipient capable of solubilising the anti-inflammatory compound and targeting said compound to the pilosebaceous ducts on application on the skin for the manufacture of a medicament intended for dermal application for the prevention or treatment of local inflammatory conditions of the skin.

In a further aspect, the invention relates to a method of preventing or treating dermal inflammatory conditions, the method comprising dermally applying at or on a site of inflammation a sufficient quantity of a pharmaceutical composition comprising a lipophilic anti-inflammatory compound and a pharmaceutically acceptable vehicle comprising a lipophilic excipient capable of solubilising the anti-inflammatory compound and targeting said compound to the pilosebaceous ducts of the skin on application.

### DETAILED DESCRIPTION OF THE INVENTION

Lipophilic Excipient

For optimum delivery of the active compound into the pilosebaceous ducts, it has been found advantageous that the lipophilic excipient has a low viscosity, i.e. a viscosity (as determined using a Haake RheoWin VT550 sensor NV (γ=700 s⁻¹)) in the range of from about 2 to about 200 mPa.s. Furthermore, the lipophilic excipient should also have a polarity similar to that of sebum, and consequently be miscible with sebum. This appears to be consistent with the results reported by T Rutherford and J G Black, British J. Dermatol. 81 (suppl. 4), 1969, p.75 disclosing a higher concentration of drug near the base of the hair follicle when the vehicle has a low polarity and low viscosity.

Without wishing to be limited to any particular theory, it would appear that the effect of the lipophilic excipient on delivery of the active compound into sebum-rich areas such as hair follicles, may be explained by the solubility properties of the excipient, as determined by Hildebrand solubility coefficients (J H Hildebrand and R L Scott, The Solubility of Non-Electrolytes, Reinhold, New York, 1949). The Hildebrand coefficients (solubility parameters δ) for model sebum compositions show that sebum is an overall non-polar, oily material with a Hildebrand coefficient of about 7.5-8 (cal/cm³)^{½} (cf. D W Osborne et al., “The Influence of Skin Surface Lipids on Topical Formulations” in Topical Drug Delivery Formulations, Vol. 42, 1990 (Drugs and the Pharmaceutical Science)). It has been found that excipients with Hildebrand coefficients within ±2 units of that of sebum are miscible with sebum and therefore suitable for the purpose of solubilising active compounds for delivery thereof to the pilosebaceous unit.

When the lipophilic excipient has surface-active properties, a favourable miscibility may alternatively be obtained by selecting a lipophilic excipient with a hydrophilic-lipophilic balance (HLB) matching the required HLB of sebum. The hydrophilic-lipophilic balance is an expression of the ratio between hydrophilic (polar) and lipophilic (non-polar) groups of a substance. The term “required HLB” is intended to indicate that, in order to be compatible with sebum, the lipophilic excipient should exhibit an HLB in a certain range (for a general discussion of required HLB, see C D Vaughan and D A Rice, J. Dispersion Science and Technology 11(1), 1990, pp. 83-91). Thus, the lipophilic excipient preferably has an HLB value in the range of from about 10 to about 18.

Finally, in a preferred embodiment the lipophilic excipient is preferably one in which the anti-inflammatory compound may be dissolved (or solubilised) so that it may exert a comedolytic effect.

In general, favourable results may be obtained when the lipophilic excipient comprises a mono-, di- or triglyceride of a C₆₋₁₂ carboxylic acid, or a vegetable oil such as fractionated coconut oil (e.g. available under the trade name Miglyol® 812).

More specifically, the lipophilic excipient may comprise a di- or triglyceride of a C₈₋₁₀ carboxylic acid.

It has been found that a particularly favourable effect is obtained when the lipophilic excipient comprises a glyceride of a C₈₋₁₀ carboxylic acid conjugated with polyalkylene glycol, in particular polyethylene glycol. For example, the lipophilic excipient may be selected from the group consisting of PEG-6 caprylic/capric glycerides (a polyethylene glycol derivative of a mixture of mono- di- or triglycerides of caprylic and capric acids with an average of 6 moles of ethylene oxide), or PEG-8 caprylic/capric glycerides (a polyethylene glycol derivative of a mixture of mono- di- or triglycerides of caprylic and capric acids with an average of 8 moles of ethylene oxide). Consistent with the general characteristics indicated above for suitable lipophilic excipients, it has, for instance, been found that the solubility parameter (δ) of PEG-8 caprylic/capric glycerides (available under the trade name Labrasol®) is about 8-9, and thus within 2 units of the solubility parameter of sebum. These lipophilic excipients typically have surface-active properties and are generally miscible with both oil and water.

While the amount of lipophilic excipient included in the present composition may vary according to the type of lipophilic excipient, it is generally preferred to include it in an amount in the range of from about 10 to about 80% w/w of the vehicle, such as in the range of from about 15 to about 75% w/w, in particular in the range of from about 25 to about 60% w/w.

Preferred Lipophilic Anti-inflammatory Compounds

Preferred lipophilic anti-inflammatory compounds included in the composition of the invention are compounds which, apart from their anti-inflammatory activity, also exhibit comedolytic activity, i.e. are capable of degrading comedones. In particular, such compounds may be selected from aminobenzophenones of the general formula I

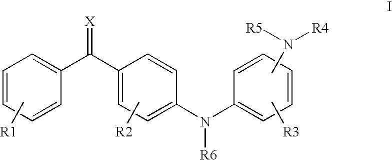

wherein

R₁ represents 1-5 substituents independently selected from the group consisting of hydrogen, halogen, hydroxy, mercapto, trifluoromethyl, amino, C₁₋₆ alkyl, C₁₋₆ alkylthio, C₁₋₆ alkylamino, C₁₋₆ alkoxy, C₁₋₆ alkoxycarbonyl, cyano, carbamoyl, phenyl and nitro;

R₂ represents 1-4 substituents independently selected from the group consisting of hydrogen, halogen, hydroxy, carboxy, mercapto, trifluoromethyl, amino, C₁₋₆ alkyl, C₁₋₆ alkylthio, C₁₋₆ alkylamino, C₁₋₆ alkoxy, C₁₋₆ alkoxycarbonyl, cyano, carbamoyl, phenyl and nitro;

R₃ represents 1-4 substituents independently selected from the group consisting of hydrogen, halogen, hydroxy, mercapto, trifluoromethyl, amino, C₁₋₆ alkyl, C₁₋₆ alkylthio, C₁₋₆ alkylamino, C₁₋₆ alkoxy, C₁₋₆ alkoxycarbonyl, cyano, carbamoyl, phenyl and carboxy;

R₄, R₅ and R₆ are independently hydrogen, trifluoromethyl, C₁₋₆ alkyl, carbamoyl, C₁₋₆ alkoxycarbonyl or C₁₋₆ alkanoyl;

X is O, S, N—OH or N—O—C₁₋₆ alkyl;

and salts thereof with pharmaceutically acceptable acids, hydrates and solvates,

with the proviso that when the compound of formula I is 4-(2-amino-4-bromophenylamino)-2-chloro-2′-methylbenzophenone, the lipophilic excipient is not PEG-8 caprylic/capric triglyceride.

As used in the specification, unless otherwise specified, the following terms have the meaning indicated:

“Alkyl” refers to any univalent group derived from an alkane by removal of a hydrogen atom from any carbon atom, and includes the subclasses of normal alkyl (n-alkyl), i.e. primary, secondary and tertiary alkyl groups respectively, and having the number of carbon atoms specified, including for example (C₁-C₅)alkyl, (C₁-C₃)alkyl, (C₁-C₂)alkyl, methyl, ethyl, n-propyl, isopropyl, n-butyl, isobutyl, sec-butyl, and t-butyl. Alkane refers to an acyclic branched or unbranched hydrocarbon having the general formula CₙH₂ₙ₊₂, and therefore consisting entirely of hydrogen atoms and saturated carbon atoms.

“Olefinic group” refers to a straight or branched acyclic hydrocarbon having one or more carbon-carbon double bonds of either E or Z stereochemistry where applicable, and having the number of carbon atoms specified. The term includes, for example, (C₂-C₁₅)olefinic group, preferably a (C₂-C₁₅)alkenyl; (C₂-C₃)olefinic group, preferably a (C₂-C₃)alkenyl; vinyl; allyl; 1-butenyl; 2-butenyl; and 2-methyl-2-propenyl. Olefinic groups having only one carbon-carbon double bond, herein called alkenyl, are preferred.

“Alkoxy” refers broadly to a radical of the formula —OR, where R is alkyl as defined above, for example (C₁-C₅)alkoxy, (C₁-C₃)alkoxy, methoxy, n-propoxy, and the like.

“Alkaloyl” refers broadly to a radical of the formula —COR where R is alkyl as defined above, for example —COCH₃ , and —COCH₂CH₃.

“Alkylthio” refers broadly to a radical of the formula —SR, where R is alkyl as defined above and includes (C₁-C₃)alkylthio, methylthio, ethylthio, n-propylthio, and 2-propylthio.

“Halogen” means the same or different of fluoro, chloro, bromo, and iodo; fluoro, chloro, and bromo being preferred.

“(C₃-C₆)cycloalkyl” means cycloalkyl groups having from 3-6 carbon atoms, and includes, for example, (C₃-C₅)cycloalkyl, cyclopropyl, cyclopentyl, and cyclohexyl.

“Carbamoyl” refers broadly to a radical of the formula —CONH₂, —CONHR, and —CONRR′ where R and R′ represent alkyl as defined above.

“Carboxy” refers broadly to a radical of the formula —COOH.

The phrase “the C-content of which can be from 1 to 5” is used herein to specify the number of carbon atoms in a substituent group, such as an alkyl group.

When R₁, R₂ and R₃ in formulae I, and II represent a phenyl group this is optionally substituted, e.g. with hydroxy; amino; nitro; cyano; halogen, preferably fluoro, chloro, or bromo; methyl; or methoxy.

Preferred compounds of formula I are compounds wherein

each R₁ is independently halogen, hydroxy, C₁₋₆ alkyl, C₁₋₆ alkylthio, C₁₋₆ alkoxy or cyano;

each R₂ is independently halogen, hydroxy, C₁₋₆ alkyl, C₁₋₆ alkylthio, C₁₋₆ alkoxy or cyano;

each R₃ is independently halogen, hydroxy, mercapto, trifluoromethyl, C₁₋₆ alkyl, C₁₋₆ alkylthio, C₁₋₆ alkoxy or cyano;

R₄ and R₅ are both hydrogen, R₆ is hydrogen or methyl; and

X is O.

Specific examples of anti-inflammatory compounds of formula I are those selected from the group consisting of

4-(2-Amino-4-bromophenylamino)-2-chloro-2′-methylbenzophenone,

4-(2-Amino-4-fluorophenylamino)-2-chloro-2′-methylbenzophenone,

4-(2-Aminophenylamino)-2,2′-dichloro-4′-methoxybenzophenone,

4′-(2-Aminophenylamino)-2,2′,3-trichloro-4-methoxybenzophenone,

4′-(2-Aminophenylamino)-2,2′,6-trichloro-4-methoxybenzophenone,

4-(2-Aminophenylamino)-2-chloro-2′-hydroxybenzophenone,

4-(2-Aminophenylamino)-2-chloro-2′-fluorobenzophenone,

4-(2-Aminophenylamino)-2,2′-dichloro-4′-hydroxybenzophenone,

4-(2-Amino-4-bromophenylamino)-2-chloro-4′-ethoxy-2′-methylbenzophenone,

4-(2-Amino-4-bromophenylamino)-2-ethoxy-2′-methylbenzophenone,

4′-(2-Aminophenylamino)-2,2′,4-trichloro-6-hydroxybenzophenone,

4-(2-Amino-5-hydroxyphenylamino)-2-chloro-2′-methylbenzophenone,

4-(2-Aminophenylamino)-2-fluoro-2′-methoxybenzophenone,

4-(2-Aminophenylamino)-2-fluoro-2′-methylbenzophenone,

4-(2-Amino-5-methoxyphenylamino)-2-chloro-2′-methylbenzophenone,

4-(2-Amino-5-chlorophenylamino)-2-chloro-2′-methylbenzophenone,

4-(2-Amino-4-trifluoromethyl)phenylamino)-2-chloro-2′-methylbenzophenone,

4-(2-Amino-3-fluorophenylamino)-2-chloro-2′-methylbenzophenone,

4-(2-Amino-N-methyl-phenylamino)-2-chloro-2′-methylbenzophenone,

4-(2-Aminophenylamino)-2,2′-dimethylbenzophenone,

4-(2-Amino-N-methyl-phenylamino)-2-chloro-2′-methylbenzophenone,

4-(2-Amino-6-methylphenylamino)-2-chloro-2′-methylbenzophenone,

4-(2-Amino-4-methoxyphenylamino)-2-chloro-2′-methylbenzophenone,

4-(2-Aminohenylamino)-2-chloro-3′-fluoro-2′-methylbenzophenone,

4-(2-Amino-4-bromophenylamino)-2-chloro-2′,3′-dimethylbenzophenone,

4-(2-Amino-4-bromophenylamino)-4′-n-butyl-2-chloro-2′-methylbenzophenone,

4-(2-Amino-4-bromophenylamino)-2,4′-dichloro-2′-methylbenzophenone,

4-(2-Amino-4-bromophenylamino)-2-fluoro-2′-methylbenzophenone,

4′-(2-Amino-4-bromophenylamino)-2′-chloro-2,4,5-trimethylbenzophenone,

4-(2Amino-4-bromophenylamino)-2-chloro-4′-fluoro-2′-methylbenzophenone,

4-(2-Amino-4-bromophenylamino)-2-chloro-2′,5′-dimethylbenzophenone,

4-(2-Amino-4-bromophenylamino)-2,3′-dichloro-2′-methylbenzophenone,

4-(2-Amino-4-bromophenylamino)-2-fluoro-4′-methoxy-2′-methylbenzophenone,

and their salts with pharmaceutically acceptable acids, their hydrates, or solvates.

Other preferred anti-inflammatory compounds for inclusion in the present composition are compounds of the general formula II

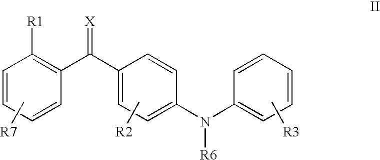

wherein

R₁ is halogen, hydroxy, mercapto, trifluoromethyl, amino, C₁₋₃ alkyl, C₁₋₃ alkylthio, C₁₋₃ alkylamino, C₁₋₃ alkoxy, C₁₋₃ alkoxycarbonyl, C₂₋₃ olefinic group, cyano, —CONH₂, phenyl or nitro;

R₂ represents 1-4 substituents independently selected from the group consisting of halogen, hydroxy, mercapto, trifluoromethyl, amino, C₁₋₃ alkyl, C₁₋₃ alkylthio, C₁₋₃ alkylamino, C₁₋₃ alkoxy, C₁₋₃ alkoxycarbonyl, C₂₋₃ olefinic group, cyano, —CONH₂, phenyl and nitro;

R₃ represents 1-5 substituents independently selected from the group consisting of halogen, hydroxy, mercapto, trifluoromethyl, amino, carboxy, carbamoyl, C₁₋₁₀ alkyl, C₁₋₁₀ alkylthio, C₁₋₁₀ alkoxy, C₁₋₁₀ alkoxycarbonyl, C₂₋₁₀ olefinic group, C₃₋₈ monocyclic hydrocarbon group, cyano and phenyl;

R₆ is hydrogen, C₁₋₆ alkyl, C₂₋₆ olefinic group, C₃₋₆ monocyclic hydrocarbon group;

R₇ represents 1-4 substituents independently selected from the group consisting of hydrogen, halogen, hydroxy, mercapto, trifluoromethyl, amino, C₁₋₃ alkyl, C₁₋₃ alkylthio, C₁₋₃ alkylamino, C₁₋₃ alkoxy, C₁₋₃ alkoxycarbonyl, C₂₋₃ olefinic group, cyano, —CONH₂, phenyl or nitro;

X is O, S or N—OH;

and salts thereof with pharmaceutically acceptable acids, hydrates or solvates.

In compounds of formula II, R₁ is preferably fluoro, chloro, bromo, hydroxy, trifluoromethyl, amino, (C₁-C₂)alkyl, (C₂-C₃)alkenyl, (C₁-C₃)alkoxy, (C₁-C₃)alkoxycarbonyl, cyano, and —CONH₂, in particular fluoro, chloro, bromo, hydroxy, methyl, or methoxy.

In compounds of formula II, each R₂ is preferably selected from the group consisting of hydrogen, fluoro, chloro, bromo, hydroxy, trifluoromethyl, amino, (C₁-C₃)alkyl, (C₂-C₃)alkenyl, and (C₁-C₃)alkoxy, in particular hydrogen, fluoro, chloro, bromo, hydroxy, methyl, and methoxy.

In compounds of formula II, each R₃ is preferably selected from the group consisting of hydrogen, fluoro, chloro, bromo, hydroxy, trifluoromethyl, (C₁-C₆)alkyl, (C₂-C₆)alkenyl, (C₁-C₆)alkoxy, (C₁-C₆)alkoxycarbonyl, cyano, carboxy, and —CONH₂, in particular hydrogen, fluoro, chloro, bromo, hydroxy, methyl, methoxy, cyano, and carboxy.

In compounds of formula II, R₆ preferably represents hydrogen, (C₁-C₄)alkyl, or (C₂-C₄)olefinic group, in particular hydrogen, methyl, or ethyl.

In compounds of formula II, X preferably represents oxygen or sulphur, in particular oxygen.

In compounds of formula II, each R₇ is preferably selected from the group consisting of hydrogen, fluoro, chloro, bromo, hydroxy, trifluoromethyl, amino, (C₁-C₂)alkyl, (C₂-C₃)alkenyl, (C₁-C₃)alkoxy, (C₁-C₃)alkoxycarbonyl, cyano, and —CONH₂, in particular hydrogen, fluoro, chloro, bromo, hydroxy, trifluoromethyl, methyl, ethyl, and methoxy.

Specific examples of anti-inflammatory compounds of formula II are selected from the group consisting of

2-[[3-Chloro-4-(2-methylbenzoyl)]phenylamino]benzonitrile,

2-Chloro-2′-methyl-4-(2-methyl-phenylamino)benzophenone,

2-Chloro-2′-methyl-4-(phenylamino)benzophenone,

2-Chloro-4-(2-methoxy-phenylamino)-2′-methylbenzophenone,

2-Chloro-4-(2-fluoro-phenylamino)-2′-methylbenzophenone,

2-Chloro-4-(2-chloro-phenylamino)-2′-methylbenzophenone,

4-(2-tert-Butoxy-phenylamino)-2-chloro-2′-methylbenzophenone,

2-Chloro-4-(2-hydroxy-phenylamino)-2′-methylbenzophenone,

2-Chloro-4-(3-chloro-phenylamino)-2′-methylbenzophenone,

2-Chloro-4-(2-[1,1,1-trifluoromethyl]-phenylamino)-2′-methylbenzophenone,

4-(4-Bromo-2,5-difluoro-phenylamino)-2-chloro-2′-methylbenzophenone,

2-Chloro-4-(2-ethyl-phenylamino)-2′-methylbenzophenone,

2-Chloro-4-(3-[1,1,1-trifluoromethyl]phenylamino)-2′-methylbenzophenone,

2-Chloro-2′-methyl-4-(2-phenyl-phenylamino)benzophenone,

2-Chloro-2′-methyl-4-(3-phenyl-phenylamino)benzophenone,

2-Chloro-4-(4-fluoro-2-methyl-phenylamino)-2′-methylbenzophenone,

2-Chloro-2′-methyl-4-(3-methyl-phenylamino)benzophenone,

2-Chloro-4-(3-methoxy-phenylamino)-2′-methylbenzophenone,

2-Chloro-4-(4-chloro-phenylamino)-2′-methylbenzophenone,

2-Chloro-2′-methyl-4-(4-phenyl-phenylamino)benzophenone,

4-(4-Bromo-phenylamino)-2-chloro-2′-methylbenzophenone,

4-(4-Bromo-3-fluoro-phenylamino)-2-chloro-2′-methylbenzophenone,

4-(2-Bromo-phenylamino)-2-chloro-2′-methylbenzophenone,

2-Chloro-4-(4-chloro-2-methyl-phenylamino)-2′-methylbenzophenone,

2-Chloro-4-(4-chloro-3-fluoro-phenylamino)-2′-methylbenzophenone,

2-Chloro-4-(3-fluoro-phenylamino)-2′-methylbenzophenone,

2-Chloro-4-(3,5-difluoro-phenylamino)-2′-methylbenzophenone,

4-(3-Bromo-phenylamino)-2-chloro-2′-methylbenzophenone,

2-Chloro-4-(3,4-difluoro-phenylamino)-2′-methylbenzophenone,

2-Chloro-4-(5-fluoro-2-methyl-phenylamino)-2′-methylbenzophenone,

2-Chloro-4-(3-fluoro-2-methyl-phenylamino)-2′-methylbenzophenone,

Ethyl 2-[[3-chloro-4-(2-methylbenzoyl)]phenylamino]benzoate,

2-Chloro-3′-fluoro-4-(4-fluoro-2-methyl-phenylamino)-2′-methylbenzophenone,

2-[[3-Chloro-4-(2-methylbenzoyl)]phenylamino]benzoic acid,

and salts thereof with pharmaceutically acceptable acids, hydrates and solvates.

The compounds of formula I may be prepared by method described in WO 01/05744, which is hereby incorporated by reference in its entirety. The compounds of formula II may be prepared by methods described in WO 01/42189, which is hereby incorporated by reference in its entirety.

The compounds of formula I and II herein may be used in the form of their salts which are formed with pharmaceutically acceptable inorganic or organic acids, such as hydrochloric, hydrobromic and hydroiodic acid, phosphoric acid, sulphuric acid, nitric acid, p-toluenesulphonic acid, methanesulphonic acid, formic acid, acetic acid, propionic acid, citric acid, tartaric acid, succinic acid, benzoic acid, maleic acid, these examples being considered as non-limiting for the invention.

Features of Acne Medication

Table 1 below summarises the various therapeutic effects and side effects of the existing drugs for treatment of acne.

TABLE 1
Features of acne medication.

| | | | | Poten- | |
|---|---|---|---|---|---|
| | Anti- | Antibac- | Comedo- | tial | Terato- |
| Agent | inflam. | terial | lytic | irritant | genic |
| Benzoyl | Maybe | Yes | Somewhat | Yes | Unknown |
| peroxide* | | | | | |
| Azelaic acid* | Maybe | Yes | Yes | Yes | No |
| Clindamycin* | Maybe | Yes | No | No | Probably |
| | | | | | not |
| Erythromycin* | Maybe | Yes | No | No | Unknown |
| Fucidin* | Maybe | Yes | No | Yes | No |
| Tretinoin* | No | No | Yes | Yes | Maybe |
| Tetracyclin** | Yes | Yes | No | No | Yes |
| Doxycyclin** | Yes | Yes | No | No | Yes |
| Minocyclin** | Yes | Yes | No | No | Yes |
| Erythromycin** | Yes | Yes | No | No | Unknown |
| Isotretinoin** | Yes | Yes | Yes | Yes | Yes |
| *Topical administration | | | | | |
| **systemic administration | | | | | |
| References to Table 1: Scientific American Medicine, Dec.96, Chapter 2 Dermatology, Acne Vulgaris and Acneiform Eruptions (E.A. Abel and E.M. Faber). Lægemiddelkataloget 1996, Acnemidler pp. 197-198, (Dansk Lægemiddelinformation A/S, Copenhagen). Models in Dermatology, vol. 2, pp. 145-158, (Maibach, Lowe), Repair and Antibacterial Effects of Topical Antiseptic Agents. Models in Dermatology, vol 2, pp 59-63, (Maibach, Lowe), Anti-Acne Activity of | | | | | |
| # Retinoids in the Rhino Mouse. Models in Dermatology, vol 2, pp. 35-42, (Maibach, Lowe), Evaluation of Topical Nonsteroidal Anti-inflammatory Agents. International Pharmacy Journal, Volume 9, No. 1, 1995, New Medicines, Acne vulgaris. Drugs 39 (5) 681-692, 1990, Current Views on the Aetiology, Pathogenesis and Treatment of Acne Vulgaris (L. Lever and R. Marks). Drugs 48 (1), 59-70, 1994, Acne, A Review of Optimum Treatment (N.L. Sykes and G.F. Webster). | | | | | |

By way of comparison, it should be noted that compounds of formula I or II included in the present composition have been found to exhibit a satisfactory antiinflammatory and comedolytic effect while, at the same time, being less irritative than, for instance, Tretinoin. Furthermore, compounds of formula I or II have not been found to exhibit any antibacterial effect (which carries the inherent risk of the development of resistance problems).

Pharmaceutical Formulation

Formulations suitable for topical administration include liquid or semi-liquid preparations such as liniments, lotions, gels, microemulsions, oil-in-water or water-in-oil emulsions such as creams, ointments or pastes; or solutions or suspensions such as drops.

In addition to the lipophilic excipient, the vehicle may comprise a solvent selected from the group consisting of water, an alcohol or mixtures thereof. When the vehicle comprises an alcohol, it is preferably selected from ethanol, propanol or isopropanol.

The vehicle may additionally comprise a penetration enhancer. The penetration enhancer may advantageously be selected from the group consisting of propylene glycol mono- or diesters of C₂₋₂₀ carboxylic acids or C₄₋₁₆ alkyl esters. Examples of suitable penetration enhancers are propylene glycol octanoate, propylene glycol decanoate, propylene glycol dipelargonate, propylene glycol monolaurate, propylene glycol monomyristate, propylene glycol dicaprylate, diethyl adipate, dicapryl adipate, diisopropyl adipate, dipropyl adipate, dioctyl adipate and dibutyl adipate.

The vehicle may additionally comprise a thickening agent or emulsifier, e.g. a carbomer. Examples of suitable thickening agents or emulsifiers include Pemulen® TR1, Pemulen® TR2, Carbopol® 1342 NF, Carbopol® 1382, Carbopol® ETD 2020, Carbopol® 980 NF, Carbopol® 940 NF, Carbopol® 974, and Carbopol® 934 P NF, Carbopol® 5984 EP, Carbopol® 2984 and Carbopol® Ultrez-10.

In a currently favoured embodiment of the present composition, the vehicle comprises

(a) a lipophilic excipient in an amount of about 10-80% w/w, such as about 10-75% w/w,

(b)an alcohol in an amount of about 0-80% w/w, such as about 10-80% w/w, e.g. about 35-70% w/w,

(c) water in an amount of about 0-50% w/w, such as about 0-40% w/w, e.g. 0-30% w/w,

(d) a penetration enhancer in an amount of about 0-15% w/w, such as about 1-10% w/w,

(e) a thickening agent in an amount of about 0-15% w/w, such as about 0.2-3% w/w such as about 0.5-1.5% w/w, and

(f) a surfactant in an amount of about 0-5% w/w, such as about 0-1%.

The amount of the individual ingredients in the composition will, to some extent, depend on the concentration of the active compound incorporated therein. Thus, at a concentration of 3% w/v of the active compound, the content of water in the composition should preferably not exceed about 40% w/w.

The amount of active compound in the present composition may vary according to the severity of the condition to be treated, but will generally be in the range of from about 0.5 to about 5% w/v, preferably from about 1 to about 4.5% w/v, more preferably from about 2 to about 4% w/v, such as about 3% w/v, relative to the amount of vehicle.

The dosage in which the anti-inflammatory compound is administered will vary between wide limits, depending on the age and condition of the patient and the discretion of the physician treating the patient. A suitable dose of the present composition comprising an antiinflammatory compound of formula I or II for topical treatment will, however, generally be in the range of about 1-3 mg/cm² administered one or more times a day.

In addition to the aforementioned ingredients, the composition of the present invention may include one or more additional ingredients, such as other therapeutically active compounds applied in the treatment of dermal inflammatory conditions, especially in the treatment of acne, for instance, azelaic acid, chlorhexidine, topical and systemic antibiotics, such as Fucidin®, clindamycin, erythromycin and tetracycline or retinoids, such as adapalene, tretinoin or isotretinoin. When the present composition is intended for the treatment of other dermal inflammatory conditions, such as dermatitis, it may include other therapeutically active components such as corticosteroids, immunosuppressants such as rapamycin or tacrolimus, or PDE4 inhibitors. Alternatively, the other active component may be present in a separate container, and each active component may be applied separately, optionally with different dosing schedules and times of administration, at the physician's discretion.

Furthermore, the present composition may comprise a topical anesthetic, e.g. benzocaine, lidocaine, bupivacaine, chlorprocaine, dibucaine, etidocaine, ketamine, pramoxine, etc.

The present composition may also comprise other components commonly used in topical formulations for application on skin, e.g. sunscreening agents, antioxidants (e.g. alpha-tocopherol), preservatives, emulsifiers, pigments, colouring agents, astringents, skin soothing agents, skin healing agents, and skin conditioning agents, such as urea, glycerol, allantoin or bisabolol, cf. CTFA Cosmetic Ingredients Handbook, 2^{nd} Ed., 1992.

The present invention is described in further detail in the following examples which are not in any way intended to limit the scope of the invention as claimed.

### EXAMPLES

### Example 1

Preparation of Dermal Formulations

Test Substance

2-chloro-4-(4-fluoro-2-methyl-phenylamino)-2′-methylbenzophenone.

Vehicle Components

Labrasol®: PEG-8 caprylic/capric glycerides

Cetiol® B: dibutyl adipate

Arlamol® DOA: dioctyl adipate

Miglyol® 812: fractionated coconut oil

Miglyol® 840: propylene glycol octanoate decanoate

DPPG®: propylene glycol dipelargonate

Pemulen® TR1: carbomer

Pemulen® TR2: carbomer

Span® 80: sorbitan oleate

Ethanol

Water

Plurol Isostearique®: polyglycerol-6-isostea rate

Test Formulations

A. A formulation was prepared by thoroughly
mixing the following vehicle components:

| | % w/w |
|---|---|
| Ethanol | 60 |
| Arlamol ® DOA | 5 |
| Labrasol ® | 25 |
| water | 10 |
| DL-alpha-tocopherol | 0.05 |

3% w/v of the test substance was weighed into a 100 ml volumetric flask. The vehicle mixture was added up to 100 ml with stirring to allow the test substance to dissolve completely in the vehicle, and the solution was filtered. The resulting formulation was stored at room temperature and protected from light.

B. A formulation was prepared by thoroughly
mixing the following vehicle components:

| | % w/w |
|---|---|
| Ethanol | 60 |
| Miglyol ® 840 | 5 |
| Labrasol ® | 25 |
| water | 10 |
| DL-alpha-tocopherol | 0.05 |

3% w/v of the test substance was weighed into a 100 ml volumetric flask. The vehicle mixture was added up to 100 ml with stirring to allow the test substance to dissolve completely in the vehicle, and the solution was filtered. The resulting formulation was stored at room temperature and protected from light.

C. A formulation was prepared by thoroughly
mixing the following vehicle components:

| | % w/w |
|---|---|
| Ethanol | 50 |
| Miglyol ® 840 | 5 |
| Labrasol ® | 35 |
| water | 10 |
| DL-alpha-tocopherol | 0.05 |

3% w/v of the test substance was weighed into a 100 ml volumetric flask. The vehicle mixture was added up to 100 ml with stirring to allow the test substance to dissolve completely in the vehicle, and the solution was filtered. The resulting formulation was stored at room temperature and protected from light.

D. A formulation was prepared by thoroughly
mixing the following vehicle components:

| | % w/w |
|---|---|
| Ethanol | 35 |
| Miglyol ® 840 | 5 |
| Labrasol ® | 50 |
| water | 10 |
| DL-alpha-tocopherol | 0.05 |

3% w/v of the test substance was weighed into a 100 ml volumetric flask. The vehicle mixture was added up to 100 ml with stirring to allow the test substance to dissolve completely in the vehicle, and the solution was filtered. The resulting formulation was stored at room temperature and protected from light.

E. A formulation was prepared by thoroughly
mixing the following vehicle components:

| | % w/w |
|---|---|
| Ethanol | 65 |
| Labrasol ® | 25 |
| water | 10 |
| DL-alpha-tocopherol | 0.05 |

3% w/v of the test substance was weighed into a 100 ml volumetric flask. The vehicle mixture was added up to 100 ml with stirring to allow the test substance to dissolve completely in the vehicle, and the solution was filtered. The resulting formulation was stored at room temperature and protected from light.

F. A formulation was prepared by thoroughly
mixing the following vehicle components:

| | % w/w |
|---|---|
| Ethanol | 55 |
| Labrasol ® | 35 |
| water | 10 |
| DL-alpha-tocopherol | 0.05 |

3% w/v of the test substance was weighed into a 100 ml volumetric flask. The vehicle mixture was added up to 100 ml with stirring to allow the test substance to dissolve completely in the vehicle, and the solution was filtered. The resulting formulation was stored at room temperature and protected from light.

G. A microemulsion was prepared by thoroughly mixing
the following vehicle components (Plurol Isostearique ® was
heated to 60-80° C. and homogenised before being
added to the vehicle mixture)

| | % w/w |
|---|---|
| Ethanol | 10 |
| Miglyol ® 840 | 10 |
| Labrasol ® | 33 |
| Plurol Isostearique ® | 17 |
| water | 30 |
| DL-alpha-tocopherol | 0.05 |

1.5% w/v of the test substance was weighed into a 100 ml volumetric flask. The vehicle mixture was added up to 100 ml with stirring to allow the test substance to be dissolved completely in the vehicle, and the solution was filtered. The resulting formulation was stored at room temperature and protected from light.

H. A microemulsion was prepared by thoroughly mixing
the following vehicle components (Plurol Isostearique ® was
heated to 60-80° C. and homogenised before being
added to the vehicle mixture)

| | % w/w |
|---|---|
| Ethanol | 10 |
| Arlamol ® DOA | 10 |
| Labrasol ® | 33 |
| Plurol Isostearique ® | 17 |
| water | 30 |
| DL-alpha-tocopherol | 0.05 |

1.5% w/v of the test substance was weighed into a 100 ml volumetric flask. The vehicle mixture was added up to 100 ml with stirring to allow the test substance to be dissolved completely in the vehicle, and the solution was filtered. The resulting formulation was stored at room temperature and protected from light.

I. A formulation was prepared by thoroughly
mixing the following vehicle components:

| | % w/w |
|---|---|
| Ethanol | 60 |
| DPPG | 5 |
| Labrasol ® | 25 |
| water | 10 |
| DL-alpha-tocopherol | 0.05 |

3% w/v of the test substance was weighed into a 100 ml volumetric flask. The vehicle mixture was added up to 100 ml with stirring to allow the test substance to dissolve completely in the vehicle, and the solution was filtered. The resulting formulation was stored at room temperature and protected from light.

J. A formulation was prepared by thoroughly
mixing the following vehicle components:

| | % w/w |
|---|---|
| Ethanol | 60 |
| Cetiol ® B | 5 |
| Labrasol ® | 25 |
| water | 10 |
| DL-alpha-tocopherol | 0.05 |

3% w/v of the test substance was weighed into a 100 ml volumetric flask. The vehicle mixture was added up to 100 ml with stirring to allow the test substance to dissolve completely in the vehicle, and the solution was filtered. The resulting formulation was stored at room temperature and protected from light.

K. A formulation was prepared by thoroughly
mixing the following vehicle components

| | % w/w |
|---|---|
| Ethanol | 65 |
| Labrasol ® | 25 |
| Pemulen ® TR1 | 0.5 |
| water | 10 |
| DL-alpha-tocopherol | 0.05 |
| Aminomethyl propanol | q.s. (to a pH of about 6.5) |

3% w/v of the test substance was dissolved in the Labrasol®. Pemulen® TR1 was dispersed by homogenisation in the Labrasol® phase. Ethanol was added to the dispersion with mixing. Water and aminomethyl propanol were mixed and slowly added to the Labrasol® phase with mixture. The resulting formulation was stored at room temperature and protected from light.

L. A formulation was prepared by thoroughly
mixing the following vehicle components

| | |
|---|---|
| Miglyol® 812 | 515 mg |
| Miglyol ® 840 | 50 mg |
| Pemulen ® TR2 | 2 mg |
| Water | 400 mg (ad 1000 mg) |
| Span ® 80 | 2 mg |
| Test substance | 30 mg |
| Aminomethyl propanol | q.s. (to a pH of 5.0-6.0) |
| DL-α-tocopherol | 0.5 mg |

The test substance was dissolved in Miglyol® 812 and Miglyol® 840. Span® 80 and DL-α-tocopherol were added to the solution (oil phase). Pemulen® TR2 was dispersed in the oil phase by homogenisation. Water, including aminomethyl propanol, was slowly added to the oil phase with mixing.

M. A formulation was prepared by
thoroughly mixing the following vehicle components

| | |
|---|---|
| Miglyol ® 812 | 565.5 mg |
| Pemulen ® TR2 | 2 mg |
| Water | 400 mg (ad 1000 mg) |
| Span ® 80 | 2 mg |
| Test substance | 30 mg |
| Aminomethyl propanol | q.s. (to a pH of 5.0-6.0) |
| DL-α-tocopherol | 0.5 mg |

The test substance was dissolved in Miglyol® 812. Span® 80 and DL-α-tocopherol were added to the solution (oil phase). Pemulen® TR2 was dispersed in the oil phase by homogenisation. Water, including aminomethyl propanol, was slowly added to the oil phase with mixing.

### Example 2

Pharmacological Methods

To study the effect of the composition of the present invention in vivo, the following procedures were used.

Skin Penetration Study

Twenty-four hairless female rats (OFA-hr/hr, IFFA CREDO, France) were used in the experiment. The animals were 8 weeks old with a mean weight of 205 g.

Two application areas were marked on the back of each rat (test area A and B). One of four test formulations (A, B, E and H as described in Example 1) containing test substance (1) labelled with ³H (specific activity: 0.26 MBq/mg) was applied to the circular test area (r=9 mm; 2.54 cm²) using a pipette. The test formulation was distributed over the test area using a glass spatula. After application, the remaining formulation was weighed and the exact amount of applied formulation was found. The target dose was 3 mg/cm² (7.62 mg/test area) for all formulations. This corresponds to 11 μl of each of formulations A, B and E, and 9 μl of formulation H.

The test areas were secured by foam pads, teflon rings and an elastic tubular net bandage (Bend-A-Rete, Artsana, Como, Italy). 24 hours later the rats were sacrificed, and the application accessories were removed. Excess formulation was removed from the test areas using cotton tampons and tape stripes. The skin of the back of each rat was cut and three 8 mm punch biopsies were taken from each test area.

Each formulation was applied to 12 test areas, 6 times to test area A (left side of the rat) and 6 times to test area B (right side of the rat). The twelve test areas were distributed on 10 rats. Eight of the rats had one formulation applied to one test area, and another formulation applied to the other test area. The remaining two rats had the same formulation applied to both test areas.

The total radioactivity in the skin was determined. The biopsies were dissolved in the tissue solubiliser Soluene 350 (Packard®) and the biopsies were analysed for content of radioactivity by liquid scintillation counting. Mean content of activity in the tree biopsies was used to calculate the total radioactivity in the entire test area. The results are given as total radioactivity relative to the applied total radioactivity in percent.

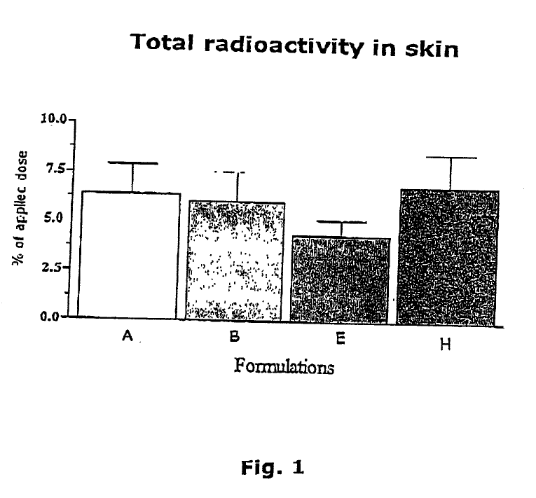

The total radioactivity measured in the skin (stratum corneum, viable epidermis and dermis) appears from FIG. 1. There is a statistically significant difference between the formulations tested. The highest skin penetration was found for formulations A and H amounting to 6.4%±1.5 and 6.9%±1.7, respectively, of the applied total radioactivity. Formulation B resulted in a significantly lower radioactivity in the skin compared to formulation H, i.e. 6.0%±1.5 of the applied dose. The lowest total radioactivity was found after application of formulation E, i.e. 4.4%±0.8 of the applied dose. The presence of a penetration enhancer (Arlamol® in formulation A and Miglyol® in formulation B) results in increased penetration of skin compared to formulation E. The two penetration enhancers appear to have a similar effect as there is no statistically significant difference between formulation A and formulation B.

Significantly lower skin levels of formulation A were observed than were found in an earlier study of the corresponding formulation without Labrasol®. The lower skin penetration of formulation A is assumed to be due to the targeting effect of Labrasol® to the pilosebaceous unit. This is confirmed by the high comedolytic activity of formulations B, C and D (see below).

Rhino Mouse Study

The rhino mouse is an in vivo model for the study of hyperplastic and comedolytic potency of various drugs used in the treatment of acne. The rhino mouse has follicles on the skin. The orifices are distended with horny material and these structures resembles human microcomedones and are referred to as utriculi or pseudocomedones.

In the present study, the comedolytic activity and skin irritation potential of 3% w/v solutions of test substance (1) in different vehicles was studied in order to select a formulation suited for topical application. Selected commercial acne products were included in the study for the purpose of comparison.

105 female rhino mice (RHJ/LeJ Rhino, hr^{rh}/hr^{rh}) aged 6-9 weeks were obtained from M&B A/S, Denmark. The mice were placed in cages with free access to a commercial rodent diet (Altromin 1324) and tap water. Groups of 6 mice were treated with each of the test formulations B, C, D, E, F and G by even application of 60 μl of one of these test formulations over the dorsal trunk once a day for 3 weeks (21 days) including weekends. As a control, groups of 6 mice were treated with the corresponding vehicle containing no test substance. For reference, groups of 6 mice were treated with Differin™ solution (containing 0.1% adapalene) and Isotrex® gel 24 (containing 0.05% isotretionin), respectively, and a group of 3 mice was treated with Redap® gel (containing 0.1% adapalene).

The clinical appearance (erythema and dryness) of the dorsal skin was observed and scored daily for individual animals within each treatment group.

The mice were killed one day after the end of the treatment period and the dorsal skin was removed. A circular (8 mm diameter) skin biopsy was taken from each animal and fixed in 10% phosphate buffered formalin. The biopsies were embedded in paraffin, and six 5 μm slices were prepared from each biopsy block and stained with hematoxylin and eosin. These slices were evaluated under the microscope (Nikon LABOPHOT-2) for the number of epidermal utriculi and utriculi profile (×200) and the epidermal thickness (×400), and the mean value and standard deviation for each animal was estimated. Thus, the effect within each group of animals was based on an evaluation of 18 to 36 slices in all dependent on the number of animals in the treatment group (3 or 6).

Two sample t-tests were performed comparing differences in mean number of utriculi between formulations containing test substance (1) and corresponding vehicles without test substance. Significance level for P<0.05.

Results

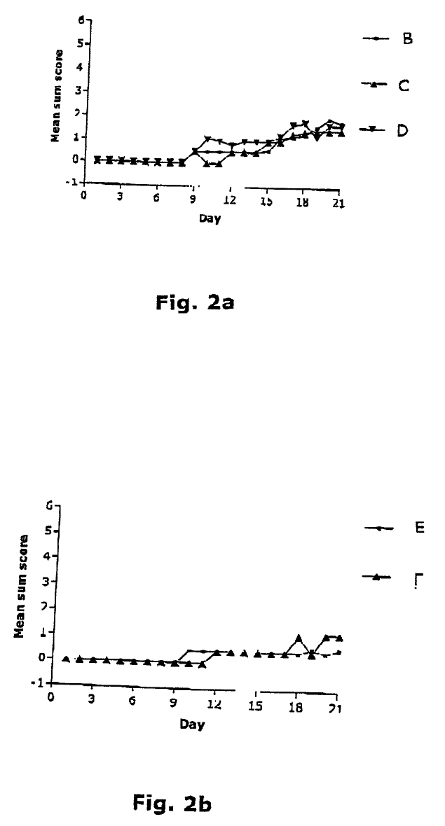

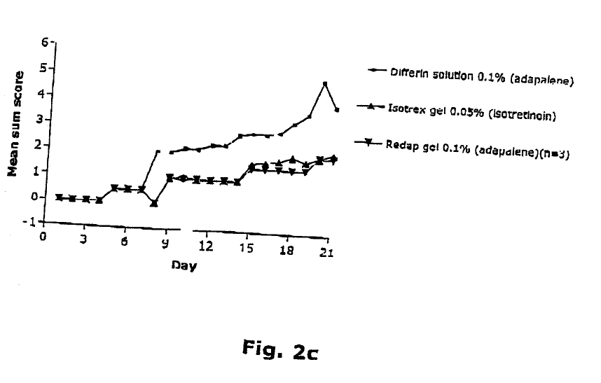

The results of the skin irritation study are shown in FIGS. 2a, 2b and 2c. It appears from FIG. 2b that formulations E and F induced only very slight skin irritation (erythema) from about treatment day 10 onwards. Formulations B and C induced slight erythema and very slight to slight dryness from about treatment day 9 onwards, as shown in FIG. 2a. It further appears from FIG. 2a that formulation D was slightly more irritative than formulations B and C.

By way of comparison, the Isotrex® gel and Redap® gel induced skin irritation within the same range and comparable to the skin irritation observed for formulation D. The Differin™ solution induced considerably more skin irritation than all the other formulations tested. Skin irritation was observed from day 5 onwards, and at the end of the treatment period, intense erythema and moderate dryness was observed for all the animals treated.

In the comedolytic activity study, formulations B, C and D induced a reduction in the number of utriculi within the range of 84-89%, and also induced 2-3 fold increases in epidermal thickness. No differences in efficacy were observed among these three formulations. The control vehicles did not have any effect on utriculi or epidermis.

Formulations E and F induced a reduction in the number of utriculi within the range of 69-78%. Epidermal thickness was 2-3 fold increased. The control vehicles did not have any effect on utriculi or epidermis.

Formulation G induced a reduction in the number of utriculi within the range of 78-85% compared to the control vehicle.

By way of comparison, the three commercial products induced a reduction in the number of utriculi in the range of 93-97% compared to untreated controls. The epidermal thickness was increased twofold.

By way of further comparison, a formulation containing test substance (1) dissolved in ethanol only induced a 51% reduction in the number of utriculi. This shows that an enhanced effect of the active substance may be obtained when it is dissolved in a vehicle containing a lipohilic excipient such as Labrasol® which is capable of targeting the active substance to the pilosebaceous unit.

1. A pharmaceutical composition for dermal application comprising a lipophilic anti-inflammatory compound and a pharmaceutically acceptable vehicle comprising a lipophilic excipient capable of solubilising the anti-inflammatory compound and targeting said compound to the pilosebaceous ducts on application of the composition to the skin.

2. A composition according to claim 1, wherein the lipophilic excipient has a viscosity in the range of from about 2 to about 200 mpa.s.

3. A composition according to claim 2, wherein the lipophilic excipient further has a polarity similar to that of sebum.

4. A composition according to any of of claims 1-3, wherein the lipophilic excipient has a Hildebrand coefficient of about 7.5-8.0±2 (cal/cm³)^{½}.

5. A composition according to any one of claims 1-4, wherein the lipophilic excipient comprises a mono-, di- or triglyceride of a C₆₋₁₂ carboxylic acid, or a vegetable oil such as fractionated coconut oil.

6. A composition according to claim 5, wherein the lipophilic excipient comprises a glyceride of a C₈₋₁₀ carboxylic acid.

7. A composition according to claim 6, wherein the lipophilic excipient comprises a glyceride of a C₈₋₁₀ carboxylic acid conjugated with polyalkylene glycol, in particular polyethylene glycol.

8. A composition according to claim 5, wherein the lipophilic excipient is selected from the group consisting of PEG-6 caprylic/capric glycerides or mixtures thereof, or PEG-8 caprylic/capric glycerides or mixtures thereof.

9. A composition according to any of claims 1-8, wherein the vehicle additionally comprises a solvent selected from the group consisting of water, an alcohol or mixtures thereof.

10. A composition according to claim 9, wherein the alcohol is ethanol, propanol or isopropanol.

11. A composition according to any of claims 1-10, wherein the anti-inflammatory compound is one which, in addition to exhibiting an anti-inflammatory activity, exhibits a comedolytic activity.

12. A composition according to any of claims 1-11, wherein the anti-inflammatory compound has the general formula I 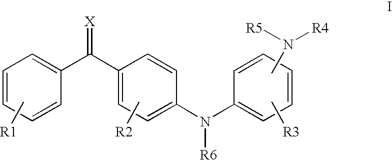
wherein
R₁ represents 1-5 substituents independently selected from the group consisting of hydrogen, halogen, hydroxy, mercapto, trifluoromethyl, amino, C₁₋₆ alkyl, C₁₋₆ alkylthio, C₁₋₆ alkylamino, C₁₋₆ alkoxy, C₁₋₆ alkoxycarbonyl, cyano, carbamoyl, phenyl and nitro;
R₂ represents 1-4 substituents independently selected from the group consisting of hydrogen, halogen, hydroxy, carboxy, mercapto, trifluoromethyl, amino, C₁₋₆ alkyl, C₁₋₆ alkylthio, C₁₋₆ alkylamino, C₁₋₆ alkoxy, C₁₋₆ alkoxycarbonyl, cyano, carbamoyl, phenyl and nitro;
R₃ represents 1-4 substituents independently selected from the group consisting of hydrogen, halogen, hydroxy, mercapto, trifluoromethyl, amino, C₁₋₆ alkyl, C₁₋₆ alkylthio, C₁₋₆ alkylamino, C₁₋₆ alkoxy, C₁₋₆ alkoxycarbonyl, cyano, carbamoyl, phenyl and carboxy;
R₄, R₅ and R₆ are independently hydrogen, trifluoromethyl, C₁₋₆ alkyl, carbamoyl, C₁₋₆ alkoxycarbonyl or C₁₋₆ alkanoyl;
X is O, S, N—OH or N—O—C₁₋₆ alkyl;
and salts thereof with pharmaceutically acceptable acids, hydrates and solvates,
with the proviso that when the compound of formula I is 4-(2-amino-4-bromophenylamino)-2-chloro-2′-methylbenzophenone, the lipophilic excipient is not PEG-8 caprylic/capric triglyceride.

13. A composition according to claim 12, wherein
each R₁ is independently halogen, hydroxy, C₁₋₆ alkyl, C₁₋₆ alkylthio, C₁₋₆ alkoxy or cyano;
each R₂ is independently halogen, hydroxy, C₁₋₆ alkyl, C₁₋₆ alkylthio, C₁₋₆ alkoxy or cyano;
each R₃ is independently halogen, hydroxy, mercapto, trifluoromethyl, C₁₋₆ alkyl, C₁₋₆ alkylthio, C₁₋₆ alkoxy or cyano;
R₄ and R₅ are both hydrogen, R₆ is hydrogen or methyl; and
X is O.

14. A composition according to claim 13, wherein the anti-inflammatory compound is selected from the group consisting of
4-(2-Amino-4-bromophenylamino)-2-chloro-2′-methylbenzophenone,
4-(2-Amino-4-fluorophenylamino)-2-chloro-2′-methylbenzophenone,
4-(2-Aminophenylamino)-2,2′-dichloro-4′-methoxybenzophenone,
4′-(2-Aminophenylamino)-2,2′,3-trichloro-4-methoxybenzophenone,
4′-(2-Aminophenylamino)-2,2′,6-trichloro-4-methoxybenzophenone,
4-(2-Aminophenylamino)-2-choro-2′-hydroxybenzophenone,
4-(2-Aminophenylamino)-2-chloro-2′-fluorobenzophenone,
4-(2-Aminophenylamino)-2,2′-dichloro-4′-hydroxybenzophenone,
4-(2-Amino-4-bromophenylamino)-2-chloro-4′-ethoxy-2′-methylbenzophenone,
4-(2-Amino-4-bromophenylamino)-2-ethoxy-2′-methylbenzophenone,
4′-(2-Aminophenylamino)-2,2′,4-trichloro-6-hydroxybenzophenone,
4-(2-Amino-5-hydroxyphenylamino)-2-chloro -2′-methylbenzophenone,
4-(2-Aminophenylamino)-2-fluoro-2′-methoxybenzophenone,
4-(2-Aminophenylamino)-2-fluoro-2′-methylbenzophenone,
4-(2-Amino-5-methoxyphenylamino)-2-chloro-2′-methylbenzophenone,
4-(2-Amino-5-chlorophenylamino)-2-chloro-2′-methylbenzophenone,
4-(2-Amino-4-(trifluoromethyl)phenylamino)-2-chloro-2′-methylbenzophenone,
4-(2-Amino-3-fluorophenylamino)-2-chloro-2′-methylbenzophenone,
4-(2-Amino-N-methyl-phenylamino)-2-chloro-2′-methylbenzophenone,
4-(2-Aminophenylamino)-2,2′-dimethylbenzophenone,
4-(2-Amino-4-fluoro-N-methyl-phenylamino)-2chloro-2′-methylbenzophenone,
4-(2-Amino-6-methylphenylamino)-2-chloro-2′-methylbenzophenone,
4-(2-Amino-4-methoxyphenylamino)-2-chloro-2′-methylbenzophenone,
4-(2-Aminohenylamino)-2-chloro-3′-fluoro-2′-methylbenzophenone,
4-(2-Amino-4-bromophenylamino)-2-chloro-2′,3′-dimethylbenzophenone,
4-(2-Amino-4-bromophenylamino)-4′-n-butyl-2-chloro-2′-methylbenzophenone,
4-(2-Amino-4-bromophenylamino)-2,4′-dichloro-2′-methylbenzophenone,
4-(2-Amino-4-bromophenylamino)-2-fluoro-2′-methylbenzophenone,
4′-(2-Amino-4-bromophenylamino)-2′-chloro-2,4,5-trimethylbenzophenone,
4-(2-Amino-4-bromophenylamino)-2-chloro-4′-fluoro-2′-methylbenzophenone,
4-(2-Amino-4-bromophenylamino)-2-chloro-2′,5′-dimethylbenzophenone,
4-(2-Amino-4-bromophenylamino)-2,3′-dichloro-2′-methylbenzophenone,
4-(2-Amino-4-bromophenylamino)-2-fluoro-4′-methoxy-2′-methylbenzophenone,
and their salts with pharmaceutically acceptable acids, their hydrates, or solvates.

15. A composition according to any of claims 1-11, wherein the anti-inflammatory compound has the general formula II 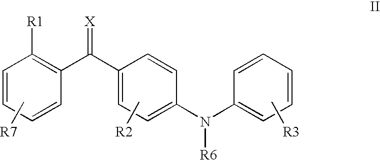
wherein
R₁ is halogen, hydroxy, mercapto, trifluoromethyl, amino, C₁₋₃ alkyl, C₁₋₃ alkylthio, C₁₋₃ alkylamino, C₁₋₃ alkoxy, C₁₋₃ alkoxycarbonyl, C₂₋₃ olefinic group, cyano, —CONH₂, phenyl or nitro;
R₂ represents 1-4 substituents independently selected from the group consisting of halogen, hydroxy, mercapto, trifluoromethyl, amino, C₁₋₃ alkyl, C₁₋₃ alkylthio, C₁₋₃ alkylamino, C₁₋₃ alkoxy, C₁₋₃ alkoxycarbonyl, C₂₋₃ olefinic group, cyano, —CONH₂, phenyl and nitro;
R₃ represents 1-5 substituents independently selected from the group consisting of halogen, hydroxy, mercapto, trifluoromethyl, amino, carboxy, carbamoyl, C₁₋₁₀ alkyl, C₁₋₁₀ alkylthio, C₁₋₁₀ alkoxy, C₁₋₁₀ alkoxycarbonyl, C₂₋₁₀ olefinic group, C₃₋₈ monocyclic hydrocarbon group, cyano and phenyl;
R₆ is hydrogen, C₁₋₆ alkyl, C₂₋₆ olefinic group, C₃₋₆ monocyclic hydrocarbon group;
R₇ represents 1-4 substituents independently selected from the group consisting of hydrogen, halogen, hydroxy, mercapto, trifluoromethyl, amino, C₁₋₃ alkyl, C₁₋₃ alkylthio, C₁₋₃ alkylamino, C₁₋₃ alkoxy, C₁₋₃ alkoxycarbonyl, C₂₋₃ olefinic group, cyano, —CONH₂, phenyl or nitro;
X is O, S or N—OH;
and salts thereof with pharmaceutically acceptable acids, hydrates or solvates.

16. A composition according to claim 15, wherein R₁ is fluoro, chloro, bromo, hydroxy, trifluoromethyl, amino, (C₁-C₂)alkyl, (C₂-C₃)alkenyl, (C₁-C₃)alkoxy, (C₁-C₃)alkoxycarbonyl, cyano, and —CONH₂, in particular fluoro, chloro, bromo, hydroxy, methyl, or methoxy.

17. A composition according to claim 15, wherein each R₂ is selected from the group consisting of hydrogen, fluoro, chloro, bromo, hydroxy, trifluoromethyl, amino, (C₁-C₃)alkyl, (C₂-C₃)alkenyl, and (C₁-C₃)alkoxy, in particular hydrogen, fluoro, chloro, bromo, hydroxy, methyl, and methoxy.

18. A composition according to claim 15, wherein each R₃ is selected from the group consisting of hydrogen, fluoro, chloro, bromo, hydroxy, trifluoromethyl, (C₁-C₆)alkyl, (C₂-C₆)alkenyl, (C₁-C₆)alkoxy, (C₁-C₆)alkoxycarbonyl, cyano, carboxy, and —CONH₂, in particular hydrogen, fluoro, chloro, bromo, hydroxy, methyl, methoxy, cyano, and carboxy.

19. A composition according to claim 15, wherein R₆ represents hydrogen, (C₁-C₄)alkyl, or (C₂-C₄)olefinic group, in particular hydrogen, methyl, or ethyl.

20. A composition according to claim 15, wherein X represents oxygen or sulphur, in particular oxygen.

21. A composition according to claim 15, wherein each R₇ is selected from the group consisting of hydrogen, fluoro, chloro, bromo, hydroxy, trifluoromethyl, amino, (C₁-C₂)alkyl, (C₂-C₃)alkenyl, (C₁-C₃)alkoxy, (C₁-C₃)alkoxycarbonyl, cyano, and —CONH₂, in particular hydrogen, fluoro, chloro, bromo, hydroxy, trifluoromethyl, methyl, ethyl, and methoxy.

22. A composition according to any of claims 15-21, wherein the anti-inflammatory compound is selected from the group consisting of
2-[[3-Chloro-4-(2-methylbenzoyl)]phenylamino]benzonitrile,
2-Chloro-2′-methyl-4-(2-methyl-phenylamino)benzophenone,
2-Chloro-2′-methyl-4-(phenylamino)benzophenone,
2-Chloro-4-(2-methoxy-phenylamino)-2′-methylbenzophenone,
2-Chloro-4-(2-fluoro-phenylamino)-2′-methylbenzophenone,
2-Chloro-4-(2-chloro-phenylamino)-2′-methylbenzophenone,
4-(2-tert-Butoxy-phenylamino)-2-chloro-2′-methylbenzophenone,
2-Chloro-4-(2-hydroxy-phenylamino)-2′-methylbenzophenone,
2-Chloro-4-(3-chloro-phenylamino)-2′-methylbenzophenone,
2-Chloro-4-(2-[1,1,1-trifluoromethyl]-phenylamino)-2′-methylbenzophenone,
4-(4-Bromo-2,5-difluoro-phenylamino)-2-chloro-2′-methylbenzophenone,
2-Chloro-4-(2-ethyl-phenylamino)-2′-methylbenzophenone,
2-Chloro-4-(3-[1,1,1-trifluoromethyl]phenylamino)-2′-methylbenzophenone,
2-Chloro-2′-methyl-4-(2-phenyl-phenylamino)benzophenone,
2-Chloro-2′-methyl-4-(3-phenyl-phenylamino)benzophenone,
2-Chloro-4-(4-fluoro-2-methyl-phenylamino)-2′-methylbenzophenone,
2-Chloro-2′-methyl-4-(3-methyl-phenylamino)benzophenone,
2-Chloro-4-(3-methoxy-phenylamino)-2′-methylbenzophenone,
2-Chloro-4-(4-chloro-phenylamino)-2′-methylbenzophenone,
2-Chloro-2′-methyl-4-(4-phenyl-phenylamino)benzophenone,
4-(4-Bromo-phenylamino)-2-chloro-2′-methylbenzophenone,
4-(4-Bromo-3-fluoro-phenylamino)-2-chloro-2′-methylbenzophenone,
4-(2-Bromo-phenylamino)-2-chloro-2′-methylbenzophenone,
2-Chloro-4-(4-chloro-2-methyl-phenylamino)-2′-methylbenzophenone,
2-Chloro-4-(4-chloro-3-fluoro-phenylamino)-2′-methylbenzophenone,
2-Chloro-4-(3-fluoro-phenylamino)-2′-methylbenzophenone,
2-Chloro-4-(3,5-difluoro-phenylamino)-2′-methylbenzophenone,
4-(3-Bromo-phenylamino)-2-chloro-2′-methylbenzophenone,
2-Chloro-4-(3,4-difluoro-phenylamino)-2′-methylbenzophenone,
2-Chloro-4-(5-fluoro-2-methyl-phenylamino)-2′-methylbenzophenone,
2-Chloro-4-(3-fluoro-2-methyl-phenylamino)-2′-methylbenzophenone,
Ethyl 2-[[3-chloro-4-(2-methylbenzoyl)]phenylamino]benzoate,
2-Chloro-3′-fluoro-4-(4-fluoro-2-methyl-phenylamino)-2′-methylbenzophenone,
2-[[3-Chloro-4-(2-methylbenzoyl)]phenylamino]benzoic acid,
and salts thereof with pharmaceutically acceptable acids, hydrates and solvates.

23. A composition according to any of claims 1-22, wherein the vehicle additionally comprises a penetration enhancer.

24. A composition according to claim 23, wherein the penetration enhancer is selected from the goup consisting of propylene glycol mono- or diesters of C₂₋₂₀ carboxylic acids or C₄₋₁₆ alkyl esters.

25. A composition according to claim 24, wherein the penetration enhancer is selected from the group consisting of propylene glycol octanoate, propylene glycol decanoate, propylene glycol dipelargonate, propylene glycol monolaurate, propylene glycol monomyristate, propylene glycol dicaprylate, diethyl adipate, dicapryl adipate, diisopropyl adipate, dipropyl adipate, dioctyl adipate and dibutyl adipate.

26. A composition according to any of claims 1-25, wherein the vehicle comprises
(a) a lipophilic excipient in an amount of about 10-80% w/w,
(b) an alcohol in an amount of about 0-80% w/w,
(c) water in an amount of about 0-50% w/w,
(d) a penetration enhancer in an amount of about 0-15% w/w,
(e) a thickening agent in an amount of about 0-15% w/w,
(f) a surfactant in an amount of about 0-5% w/w.

27. A composition according to claim 26, wherein the amount of active substance is in the range of from about 0.5 to about 5% w/v, preferably from about 1 to about 4.5% w/v, more preferably from about 2 to about 4% w/v, such as about 3% w/v, relative to the amount of vehicle.

28. Use of a pharmaceutical composition comprising a lipophilic anti-inflammatory compound and a pharmaceutically acceptable vehicle comprising a lipophilic excipient capable of solubilising the anti-inflammatory compound and targeting said compound to the pilosebaceous ducts of the skin on application for the manufacture of a medicament intended for dermal application for the prevention or treatment of local inflammatory conditions of the skin.

29. The use of claim 28, wherein the inflammatory condition is contact dermatitis, atopic dermatitis, eczema or acne.

30. Use of a pharmaceutical composition according to any of claims 1-27 for the manufacture of a medicament for exerting a comedolytic activity when applied on skin.

31. A method of preventing or treating dermal inflammatory conditions, the method comprising dermally applying at or on a site of inflammation a sufficient quantity of a pharmaceutical composition comprising a lipophilic anti-inflammatory compound and a pharmaceutically acceptable vehicle comprising a lipophilic excipient capable of solubilising the anti-inflammatory compound and targeting said compound to the pilosebaceous ducts of the skin on application.

32. The method of claim 34, wherein the inflammatory condition is contact dermatitis, atopic dermatitis, eczema or acne.
